(19) United States
(12) Patent Application Publication
(10) Pub. No.: US 2006/0034903 A1
(43) Pub. Date: Feb. 16, 2006

(54) Apparatus and method for transdermal delivery of natriuretic peptides

(76) Inventors: Yuh-Fun Maa, Millbrae, CA (US); Scott Sellers, San Mateo, CA (US); Peter Daddona, Menlo Park, CA (US); Marika Kamberi, San Jose, CA (US); Vidhya Gopalakrishnan, San Jose, CA (US); B. Michael Silber, Palo Alto, CA (US); Frank Stonebanks, Wayne, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/201,625

(22) Filed: Aug. 10, 2005

Related U.S. Application Data
(60) Provisional application No. 60/600,560, filed on Aug. 11, 2004.

(51) Int. Cl. A61K 38/17; A61M 31/00; A61F 13/02

(52) U.S. Cl. 424/448; 514/12; 604/500

(57) ABSTRACT

An apparatus and method for transdermally delivering a natriuretic peptide comprising a delivery system having a microprojection member that includes a plurality of microprojections (or array thereof) that are adapted to pierce through the stratum corneum into the underlying epidermis layer, or epidermis and dermis layers. In one embodiment, the natriuretic peptide is contained in a biocompatible coating that is applied to the microprojection member. In a further embodiment, the delivery system includes a natriuretic peptide-containing hydrogel formulation. In an alternative embodiment, the natriuretic peptide is contained in both the coating and the hydrogel formulation. In yet another embodiment, the natriuretic peptide is contained in a solid state formulation.

### CROSS-REFERENCE TO RELATED APPLICATIONS

This application claims the benefit of U.S. Provisional Application No. 60/600,560, filed Aug. 10, 2004.

### FIELD OF THE PRESENT INVENTION

The present invention relates generally to transdermal agent delivery systems and methods. More particularly, the invention relates to an apparatus and method for transdermal delivery of natriuretic peptides.

### BACKGROUND OF THE INVENTION

It is well known that acute heart failure is the single most common cause of hospitalization in the United States for patents 65 years of age and older. Indeed, acute heart failure results in approximately one million hospitalizations each year.

Nesiritide, a recombinant form of human B-type natriuretic peptide (hBNP), is often used to treat patients with acute congestive heart failure who have dyspnea (i.e., shortness of breath) at rest or with minimal activity. The noted peptide, hBNP, is a naturally occurring protein that is secreted by the heart in response to acute heart failure, e.g., when the heart is unable to pump blood efficiently, hBNP is produced.

Details of the natriuretic peptide hBNP and other brain natriuretic peptides (BNPs) and recombinant techniques for production of same are set forth in U.S. Pat. Nos. 5,114,923 and 5,674,710. The noted patents are expressly incorporated herein in their entirety.

Recent studies indicate that hBNP provides a number of additional physiologic (or therapeutic) effects, such as relaxation of blood vessels, (i.e., vasodilation), enhancing the excretion of sodium (i.e., natriuresis) and fluid (i.e., diuresis) and decreasing neurohormones (i.e, endothelin, aldosterone, angiutensin II). All of the noted physiologic effects (or actions) work in concert on the vessels, heart and kidney to decrease the fluid load on the heart, which improves cardiac performance.

Recent studies have also demonstrated a role for BNP in blocking TGF-B medicated cardiac fibroblast proliferation and myocardial fibrosis. Additional evidence further suggests an ability to inhibit cardiac remodeling after myocardial infarction.

Nesiritide's diuretic and potentially anti-fibrotic effects have also led to significant interest in its potential to address acute and chronic kidney disease. Historical exploration of BNP has demonstrated a potential-long term benefit from chronic administration in slowing disease progression towards ESRD and dialysis reliance.

At present, hBNP is only administered via intravenous (e.g., intravenous infusion), intranasal and oral transmucosal routes. Unfortunately, many active agents, such as hBNP, have reduced efficacy when orally administered, since they either are not fully absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure which sometimes results in poor patient compliance.

Transdermal delivery is thus a viable alternative for administering active agents, particularly, hBNP, that would otherwise need to be delivered via hypodermic injection or intravenous infusion. The word “transdermal”, as used herein, is a generic term that refers to delivery of an active agent (e.g., a therapeutic agent, such as a human brain natriuretic peptide or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery thus includes intracutaneous, intradermal and intraepidermal delivery via passive diffusion as well as delivery based upon external energy sources, such as electricity (e.g., iontophoresis) and ultrasound (e.g., phonophoresis).

Passive transdernal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves mechanically penetrating the outermost skin layer(s) to create micropathways in the skin. There have been many techniques and devices developed to mechanically penetrate or disrupt the outermost skin layers to create pathways into the skin. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is also possible to have the active agent that is to be delivered coated on the microprojections instead of contained in a physical reservoir. This eliminates the necessity of a separate physical reservoir and developing an agent formulation or composition specifically for the reservoir.

As stated, hBNP is at present delivered solely via intraneous routes. It would thus be desirable to provide an agent delivery system that facilitates intracutaneous administration of hBNP as well as other natriuretic peptides.

It is therefore an object of the present invention to provide a transdermal agent delivery apparatus and method that provides intracutaneous delivery of natriuretic peptides to a patient.

It is another object of the invention to provide a transdermal agent delivery apparatus and method that provides rapid on-set with tolerable Cmax.

It is another object of the invention to provide a transdermal agent delivery apparatus and method that provides biological action of hBNP for a period in the range of 2-6 hours.

It is another object of the invention to provide a transdermal agent delivery apparatus and method that can be employed once or twice daily.

It is another object of the invention to provide a natriuretic peptide-based formulation having enhanced stability for intracutaneous delivery to a patient.

It is another object of the present invention to provide a transdermal agent delivery apparatus and method that includes microprojections coated with a biocompatible coating that includes at least one natriuretic peptide, preferably, hBNP.

Another object of the present invention is to provide a transdermal agent delivery apparatus and method that includes a gel pack adapted to receive a hydrogel formulation that contains at least one natriuretic peptide, preferably, hBNP.

It is yet another object of the present invention to provide a transdermal agent delivery apparatus and method that includes a solid state form of at least one natriuretic peptide, preferably, hBNP, that is adapted to be reconstituted prior to delivery by a hydrogel.

### SUMMARY OF THE INVENTION

In accordance with the above objects and those that will be mentioned and will become apparent below, the apparatus and method for transdermally delivering a natriuretic peptide in accordance with this invention generally comprises a delivery system having a microprojection member (or assembly) that includes a plurality of microprojections (or array thereof) that are adapted to pierce through the stratum corneum into the underlying epidermis layer, or epidermis and dermis layers. In one embodiment, the microprojection member includes a biocompatible coating having at least one natriuretic peptide. In another embodiment, the microprojection member includes a hydrogel formulation having at least one natriuretic peptide. In yet another embodiment, the microprojection member includes a solid state formulation having at least one natriuretic peptide and a hydrating hydrogel formulation.

The apparatus and method provides intracutaneous administration of natriuretic peptides with improved pharmacokinetics, including rapid on-set with tolerable Cₘₐₓ and biological action of the natriuretic peptide(s) for a period of 2-6 hours.

In one embodiment of the invention, the microprojection member has a microprojection density of at least approximately 10 microprojections/cm², more preferably, in the range of at least approximately 200-2000 microprojections/cm².

In one embodiment, the microprojection member is constructed out of stainless steel, titanium, nickel titanium alloys, or similar biocompatible materials.

In another embodiment, the microprojection member is constructed out of a non-conductive material, such as polymeric materials.

Alternatively, the microprojection member can be coated with a non-conductive material, such as Parylene®, or a hydrophobic material, such as Teflon®, silicon or other low energy material.

The coating formulations applied to the microprojection member to form solid biocompatible coatings can comprise aqueous and non-aqueous formulations. In at least one embodiment of the invention, the formulation(s) includes at least one natriuretic peptide, which can be dissolved within a biocompatible carrier or suspended within the carrier.

Preferably, the natriuretic peptide is selected from the family comprising artrial natriuretic peptides (ANP), B-type or brain natriuretic peptides (BNP), C-type natriuretic peptides (CNP) and urodilatins, and analogs, active fragments, degradation products, salts, variants, simple derivatives and combinations thereof. In a preferred embodiment, the natriuretic peptide comprises a B-type natriuretic peptide (BNP), more preferably, hBNP (1-32).

In one embodiment of the invention, the natriuretic peptide comprises in the range of approximately 1-30 wt. % of the coating formulation.

Preferably, the amount of the natriuretic peptide contained in the coating formulation is in the range of approximately 1-2000 μg.

Preferably, the pH of the coating formulation is below approximately pH 9. More preferably, the pH of the coating formulation is in the range of approximately pH 3-pH 8. Even more preferably, the pH of the coating formulation is in the range of approximately pH 4-pH 6.

In one embodiment of the invention, the coating formulation includes at least one buffer. Examples of suitable buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, and alkoxylated alcohols, such as laureth-4.

In one embodiment of the invention, the concentration of the surfactant is in the range of approximately 0.001-2 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hyroxyethyl starch, dextran, poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 0.01-20 wt. %, more preferably, in the range of approximately 0.03-10 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose, stachyose, mannitol and like sugar alcohols.

Preferably, the concentration of the biocompatible carrier in the coating formulation is in the range of approximately 2-70 wt. %, more preferably, in the range of approximately 5-50 wt. % of the coating formulation.

In another embodiment, the coating formulation includes a stabilizing agent, which can comprise, without limitation, a non-reducing sugar, polysaccharide or a reducing sugar.

Suitable non-reducing sugars include, for example, sucrose, trehalose, stachyose and raffinose.

Suitable polysaccharides include, for example, dextran, soluble starch, dextrin, and insulin.

Suitable reducing sugars include, for example, monosaccharides such as apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as primeverose, vicianose, rutinose, scillabiose, cellobiose, gentiobiose, lactose, lactulose, maltose, melibiose, sophorose, and turanose, and the like.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors comprise epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. The most preferred solubilising/complexing agents comprise beta-cyclodextrin, hydroxypropyl beta-cyclodextrin, 2-hydroxypropyl-beta-Cyclodextrin and sulfobutylether7 beta-cyclodextrin.

The concentration of the solubilising/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent comprises in the range of approximately 1 wt. % to 50 wt. % of the coating formulation.

Preferably, the coating formulations have a viscosity less than approximately 500 centipoise and greater than 3 centipose.

In one embodiment of the invention, the thickness of the biocompatible coating is less than 25 microns, more preferably, less than 10 microns, as measured from the microprojection surface.

In a further embodiment of the invention, the delivery system includes hydrogel formulation. Preferably, the hydrogel formulation is contained in a gel pack.

In at least one embodiment of the invention, the hydrogel formulation contains at least one natriuretic peptide.

In a preferred embodiment, the natriuretic peptide comprises in the range of approximately 0.1-2 wt. % of the hydrogel formulation.

Preferably, the pH of the hydrogel formulation is below approximately pH 6. More preferably, the pH of the hydrogel formulation is in the range of approximately pH 3-pH 6. Even more preferably, the pH of the hydrogel formulation is in the range of approximately pH 4-pH 6.

In one embodiment of the invention, the hydrogel formulation includes at least one of the aforementioned buffers.

Preferably, the hydrogel formulations comprise water-based hydrogels having macromolecular polymeric networks.

In a preferred embodiment of the invention, the polymeric network comprises, without limitation, hyroxyethyl starch, dextran, hydroxyethylcellulose (HEC), hydroxypropylmethylcellulose (HPMC), hydroxypropycellulose (HPC), methylcellulose (MC), hydroxyethyl-methylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone) and pluronics.

The hydrogel formulation preferably includes at least one surfactant, which can be zwitterionic, amphoteric, cationic, anionic or nonionic.

In one embodiment of the invention, the surfactant comprises sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, and alkoxylated alcohols, such as laureth-4.

In another embodiment, the hydrogel formulation includes polymeric materials or polymers having amphiphilic properties, which can comprise, without limitation, cellulose derivatives, such as hydroxyethylcellulose (HEC), hydroxypropyl-methylcellulose (HPMC), hydroxypropycellulose (HPC), methylcellulose (MC), hydroxyethylmethylcellulose (HEMC) and ethylhydroxyethylcellulose (EHEC), as well as pluronics.

In a further embodiment of the invention, the hydrogel formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl-beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. The most preferred solubilising/complexing agents comprise beta-cyclodextrin, hydroxypropyl-beta-Cyclodextrin, 2-hydroxypropyl-beta-Cyclodextrin and sulfobutylether7 beta-cyclodextrin.

In another embodiment of the invention, the hydrogel formulation includes at least one non-aqueous solvent, such as ethanol, isopropanol, methanol, propanol, butanol, propylene glycol, dimethyl sulphoxide and polyethylene glycol 400. Preferably, the non-aqueous solvent comprises in the range of approximately 1 wt. % to 50 wt. % of the hydrogel formulation.

In a further embodiment of the invention, the hydrogel formulation contains at least one pathway patency modulator, which can comprise, without limitation, osmotic agents (e.g., sodium chloride), zwitterionic compounds (e.g., amino acids), and anti-inflammatory agents, such as betamethoasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinate sodium salt, paramethasone disodium phosphate and prednisolone 21-succinate sodium salt, and anticoagulants, such as citric acid, citrate salts (e.g., sodium citrate), dextrin sulfate sodium and EDTA.

In yet another embodiment of the invention, the hydrogel formulation includes at least one vasoconstrictor, which can comprise, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropolamine, propylhexedrine, pseudoephedrine, tetrahydrozoline, tramazoline, tuaminoheptane, tymazoline, vasopressin and xylometazoline, and the mixtures thereof.

In accordance with yet another embodiment of the invention, the delivery system for delivering a natriuretic peptide includes a microprojection member having top and bottom surfaces, a plurality of openings that extend through the microprojection member and a plurality of microprojections that project from the bottom surface of the microprojection member. The microprojection member further includes a hydrogel formulation and a solid state formulation having at least one natriuretic peptide, preferably, hBNP(1-32).

In one embodiment, the solid state formulation is disposed proximate the top surface of the microprojection member. In another embodiment, the solid state formulation is disposed proximate the bottom surface of the microprojection member.

In one embodiment of the invention, the hydrogel formulation is devoid of a natriuretic peptide and, hence, is merely a hydration mechanism.

In one embodiment, the solid state formulation comprises a solid film. Preferably, the solid film is made by casting a liquid formulation consisting of at least one natriuretic peptide, a polymeric material, such as hyroxyethyl starch, dextran, hydroxyethylcellulose (HEC), hydroxypropylmethylcellulose (HPMC), hydroxypropycellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone) and pluronics, a plasticizing agent, such as glycerol, propylene glycol, and polyethylene glycol, a surfactant, such as Tween 20 and Tween 80, and a volatile solvent, such as water, isopropanol, methanol and ethanol.

In one embodiment, the liquid formulation used to produce the solid film comprises: 0.1-20 wt. % natriuretic peptide, 5-40 wt. % polymer, 5-40 wt. % plasticizer, 0-2 wt. % surfactant, and the balance comprising volatile solvent.

More preferably, the concentration of the natriuretic peptide in the liquid formulation used to produce the solid film at a concentration in the range of approximately 0.1-2 wt. %.

In further embodiments of the invention, the solid state formulation is formed by a process selected from the group consisting of spray drying, freeze drying, spray freeze drying and supercritical fluid extraction. A currently preferred process is spray freeze drying. In the noted embodiments, the biocompatible coating is adapted to be reconsitituted by a suitable solvent in up to approximately 15 min, and preferably, in up to approximately 1 min. The coating formulation also preferably includes an antioxidant.

Preferably, the pH of the liquid formulation used to produce the solid state formulation is below about pH 6. More preferably, the pH of the formulation used to produce the solid state formulation is in the range of approximately pH 3-pH 6. Even more preferably, the pH of the liquid formulation used to produce the solid state formulation is in the range of approximately pH 4-pH 6.

In another embodiment, the liquid formulation used to produce the solid state formulation includes a stabilizing agent, which can comprise, without limitation, a non-reducing sugar, a polysaccharide or a reducing sugar.

Suitable non-reducing sugars include, for example, sucrose, trehalose, stachyose, or raffinose.

Suitable polysaccharides include, for example, dextran, soluble starch, dextrin, and inulin.

Suitable reducing sugars include, for example, monosaccharides, such as apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides, such as primeverose, vicianose, rutinose, scillabiose, cellobiose, gentiobiose, lactose, lactulose, maltose, melibiose, sophorose, and turanose, and the like.

In one embodiment of the invention, the liquid formulation used to produce the solid state formulation includes at least one of the aforementioned buffers.

In another embodiment of the invention, the liquid formulation used to produce the solid state formulation includes at least one of the aforementioned complexing/solubilising agents.

In a further embodiment of the invention, the liquid formulation used to produce the solid state formulation includes at least one of the aforementioned vasoconstrictors.

In a further embodiment of the invention, the liquid formulation used to produce the solid state formulation includes at least one of the aforementioned pathway patency modulators.

In accordance with one embodiment of the invention, the method for delivering a natriuretic peptide to a patient includes the following steps: (i) providing a delivery system having a microprojection member, the microprojection member including a plurality of microprojections and a biocompatible coating having at least one natriuretic peptide, (ii) applying the coated microprojection member to the patient's skin via an actuator, whereby the microprojections pierce the skin and the agent-containing coating is dissolved by body fluid and released into the skin.

The coated microprojection member is preferably left on the skin for a period lasting from 5 seconds to 24 hours. Following the desired wearing time, the microprojection member is removed from the skin.

In accordance with a further embodiment of the invention, the method for delivering a natriuretic peptide to a patient includes the following steps: (i) providing a delivery system having a microprojection member and a gel pack including a hydrogel formulation having at least one natriuretic peptide, (ii) applying the microprojection member to the patient's skin via an actuator, whereby the microprojections pierce the stratum corneum and form a plurality of microslits in the stratum corneum, and (iii) placing the gel pack on top of the applied microprojection member, whereby the hydrogel formulation migrates into and through the microslits formed by the microprojections.

The microprojection member-gel pack assembly is preferably left on the skin for a period lasting from 5 minutes to 24 hours. Following the desired wearing time, the microprojection member-gel pack assembly is removed from the skin.

In a further aspect of the noted embodiment, the microprojection member includes an agent-containing biocompatible coating and the hydrogel formulation is devoid of a natriuretic peptide and, hence, is merely a hydration mechanism.

In accordance with another embodiment of the invention, the method for delivering a natriuretic peptide includes the following steps: (i) providing a delivery system having a microprojection member and a gel pack including a hydrogel formulation having at least one natriuretic peptide, (ii) applying the microprojection member to the patient's skin via an actuator, whereby the microprojections pierce the stratum corneum and form a plurality of microslits in the stratum corneum, (iii) removing the microprojection member from the patient's skin, and (iv) placing the gel pack on top of the pretreated skin, whereby the hydrogel formulation migrates into and through the microslits formed by the microprojections.

The gel pack is preferably left on the skin for a period lasting from 5 minutes to 24 hours. Following the desired wearing time, the gel pack is removed from the skin.

In a further embodiment of the invention, the method for delivering a natriuretic peptide includes the following steps: (i) providing a delivery system having a microprojection member, a hydrogel formulation and a solid state formulation having at least one natriuretic peptide, and (ii) applying the microprojection member to the patient's skin via an actuator, whereby the microprojections pierce the stratum corneum, the hydrogel formulation hydrates and releases the agent formulation from the solid state formulation and the agent formulation migrates into and through the microslits in the stratum corneum formed by the microprojections.

The microprojection member is preferably left on the skin for a period lasting from 5 seconds to 24 hours. Following the desired wearing time, the microprojection member is removed from the skin.

In yet another embodiment of the invention, the microprojection member having a natriuretic peptide-containing biocompatible coating is applied to the patient's skin, a gel pack having a natriuretic peptide-containing hydrogel formulation is then placed on top of the applied microprojection member, whereby the hydrogel formulation and coating migrates into and through the microslits in the stratum corneum formed by the microprojections. The microprojection member-gel pack assembly is preferably left on the skin for a period lasting 5 minutes to 24 hours, more preferably, 1-6 hours. Following the desired wearing time, the microprojection member and gel pack are removed.

Preferably, the dose of natriuretic peptide delivered intracutaneously via the aforementioned natriuretic peptide methods is in the range of approximately 10-2000 μg/day, more preferably, in the range of approximately 10-1000 μg/day.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection member;

FIG. 2 is a perspective view of the microprojection member shown in FIG. 1 having a coating deposited on the microprojections, according to the invention;

FIG. 3 is a side sectional view of a microprojection member having an adhesive backing;

FIG. 4 is an exploded perspective view of one embodiment of a gel pack of a microprojection system;

FIG. 5 is an exploded perspective view of one embodiment of a microprojection member of a microprojection system;

FIG. 6 is a perspective view of one embodiment of a microprojection assembly comprising the gel pack shown in FIG. 4 and the microprojection member shown in FIG. 5;

FIG. 7 is a side sectional view of a retainer having a microprojection member disposed therein;

FIG. 8 is a perspective view of the retainer shown in FIG. 7;

FIG. 9 is an exploded perspective view of an applicator and retainer;

FIG. 10 is a graph illustrating the charge profile for hBNP (1-32);

FIG. 11 is a graph of hBNP content in a coating formulation of the invention as a function of the number of coating applications;

FIG. 12 shows SEM images of coated microprojection arrays, according to the invention;

FIG. 13 is a graph comparing plasma concentration of hBNP following transdermal and intravenous delivery, according to the invention;

FIG. 14 is a graph comparing pharmacokinetic and pharmacodynamic response following transdermal delivery of hBNP, according to the invention; and

FIG. 15 is a graph comparing pharmacodynamic response following transdermal and intravenous delivery of hBNP, according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a peptide” includes two or more such peptides; reference to “a microprojection” includes two or more such microprojections and the like.

### DEFINITIONS

The term “transdermal”, as used herein, means the delivery of an agent into and/or through the skin for local or systemic therapy. The term “transdermal” thus means and includes intracutaneous, intradermal and intraepidermal delivery of an agent, such as a peptide, into and/or through the skin via passive diffusion as well as energy-based diffusional delivery, such as iontophoresis and phonophoresis.

The term “transdermal flux”, as used herein, means the rate of transdermal delivery.

The term “natriuretic peptide”, as used herein, thus means a peptide that exhibits natriuretic activity. The term “natriuretic peptide” thus includes artrial natriuretic peptides (ANP), brain or B-type natriuretic peptides (BNP), C-type natriuretic peptides (CNP), urodilatins and peptides analogous thereto, and analogs, active fragments, degradation products, salts, variants, derivatives and combinations thereof.

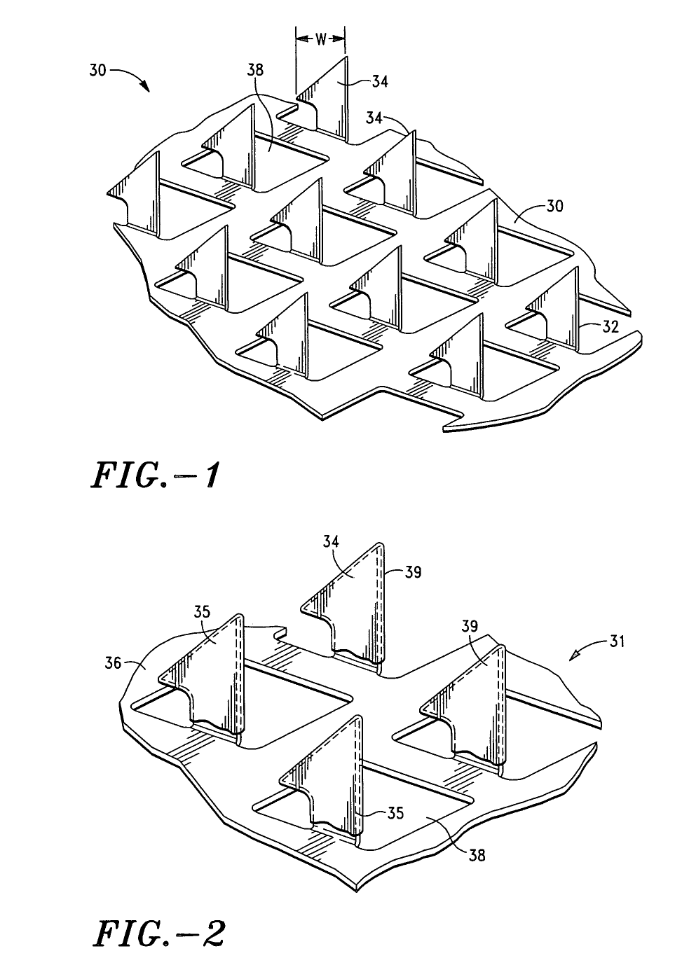

The term “brain natriuretic peptide (BNP)”, as used herein, refers to an amino acid sequence that is encoded by a DNA capable of hybridizing to an effective portion of the DNA shown in FIG. 1 of U.S. Pat. No. 5,674,710 and which has natriuretic activity.

The terms “Nesiritide” and “hBNP”, as used herein, refer to a recombinant form of human B-type natriuretic peptide, peptides analogous thereto and active fragments thereof. The terms thus include, without limitation, hBNP(1-32).

The term “co-delivering”, as used herein, means that a supplemental agent(s) is administered transdermally either before the natriuretic peptide is delivered, before and during transdermal flux of the natriuretic peptide, during transdermal flux of the natriuretic peptide, during and after transdermal flux of the natriuretic peptide, and/or after transdermal flux of the natriuretic peptide. Additionally, two or more natriuretic peptides may be formulated in the coatings and/or hydrogel formulation, resulting in co-delivery of the natriuretic peptides.

It is to be understood that more than one natriuretic peptide can be incorporated into the agent source, formulations, and/or coatings and/or solid state formulations of this invention, and that the use of the term “natriuretic peptide” in no way excludes the use of two or more such peptides.

The term “microprojections”, as used herein, refers to piercing elements which are adapted to pierce or cut through the stratum corneum into the underlying epidermis layer, or epidermis and dermis layers, of the skin of a living animal, particularly, a mammal and, more particularly, a human.

In one embodiment of the invention, the piercing elements have a projection length less than 1000 microns. In a further embodiment, the piercing elements have a projection length of less than 500 microns, more preferably, less than 250 microns. The microprojections further have a width (designated “W” in FIG. 1) in the range of approximately 25-500 microns and a thickness in the range of approximately 10-100 microns. The microprojections may be formed in different shapes, such as needles, blades, pins, punches, and combinations thereof.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 1. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture that is employed to coat the microprojections and/or arrays thereof. The natriuretic peptide, if disposed therein, can be in solution or suspension in the formulation.

The term “biocompatible coating” and “solid coating”, as used herein, is meant to mean and include a “coating formulation” in a substantially solid state.

The term “solid state formulation”, as used herein, is meant to mean and include solid films formed by casting, and powders or cakes formed by spray drying, freeze drying, spray freeze drying and supercritical fluid extraction.

As indicated above, the present invention generally comprises a delivery system including microprojection member (or system) having a plurality of microprojections (or array thereof) that are adapted to pierce through the stratum corneum into the underlying epidermis layer, or epidermis and dermis layers. The microprojection member (or system) includes at least one agent source or agent delivery medium (i.e., biocompatible coating, hydrogel formulation, solid state formulation).

As discussed in detail herein, the microprojection system provides intracutaneous administration of natriuretic peptides with improved pharmacokinetics. The improved pharmacokinetics includes rapid on-set with tolerable Cₘₐₓ and sustained biological action of the natriuretic peptide for a period in the range of 2-6 hours.

A further advantage of the present invention is that the formulations employed as and to produce the delivery mediums substantially inhibit oxidation of the natriuretic peptide(s) disposed therein. The stability of the agent-containing medium is thus significantly enhanced.

Referring now to FIG. 1, there is shown one embodiment of a microprojection member 30 for use with the present invention. As illustrated in FIG. 1, the microprojection member 30 includes a microprojection array 32 having a plurality of microprojections 34. The microprojections 34 preferably extend at substantially a 90° angle from the sheet, which in the noted embodiment includes openings 38.

According to the invention, the sheet 36 can be incorporated into a delivery patch, including a backing 40 for the sheet 36, and can additionally include adhesive 16 for adhering the patch to the skin (see FIG. 3). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², more preferably, in the range of at least approximately 200-2000 microprojections/cm². Preferably, the number of openings per unit area through which the agent passes is at least approximately 10 openings/cm² and less than about 2000 openings/cm².

As indicated, the microprojections 34 preferably have a projection length less than 1000 microns. In one embodiment, the microprojections 34 have a projection length of less than 500 microns, more preferably, less than 250 microns. The microprojections 34 also preferably have a width in the range of approximately 25-500 microns and thickness in the range of approximately 10-100 microns.

To enhance the biocompatibility of the microprojection member 30 (e.g., minimize bleeding and irritation following application to the skin of a subject), in a further embodiment, the microprojections 34 preferably have a length less than 145 μm, more preferably, in the range of approximately 50-145 μm, even more preferably, in the range of approximately 70-140 μm. Further, the microprojection member 30 comprises an array preferably having a microprojection density greater than 100 microprojections/cm², more preferably, in the range of approximately 200-3000 microprojections/cm².

The microprojection member 30 can be manufactured from various metals, such as stainless steel, titanium, nickel titanium alloys, or similar biocompatible materials.

According to the invention, the microprojection member 30 can also be constructed out of a non-conductive material, such as a polymer.

Alternatively, the microprojection member can be coated with a non-conductive material, such as Parylene®, or a hydrophobic material, such as Teflon® silicon or other low energy material. The noted hydrophobic materials and associated base (e.g., photoreist) layers are set forth in U.S. Application No. 60/484,142, which is incorporated by reference herein.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the natriuretic peptide to be administered to a host can be contained in a biocompatible coating that is disposed on the microprojection member 30 or contained in a hydrogel formulation or contained in both the biocompatible coating and the hydrogel formulation.

In a further embodiment, wherein the microprojection member includes an agent-containing solid state formulation, the natriuretic peptide can be contained in the biocompatible coating, hydrogel formulation or solid state formulation, or in all three delivery mediums.

According to the invention, at least one natriuretic peptide is contained in at least one of the aforementioned delivery mediums. The amount of the natriuretic peptide that is employed in the delivery medium and, hence, microprojection system will be that amount necessary to deliver a therapeutically effective amount of the natriuretic peptide to achieve the desired result. In practice, this will vary widely depending upon the particular natriuretic peptide, the site of delivery, the severity of the condition, and the desired therapeutic effect.

In one embodiment, the microprojection member includes a biocompatible coating that contains at least one natriuretic peptide, preferably, hBNP(1-32). Upon piercing the stratum corneum layer of the skin, the natriuretic peptide-containing coating is dissolved by body fluid (intracellular fluids and extracellular fluids such as interstitial fluid) and released into the skin (i.e., bolus delivery) for systemic therapy. Preferably, the total dose of natriuretic peptide delivered intracutaneously is in the range of approximately 10-2000 μg/day, more preferably, 10-1000 μg/day.

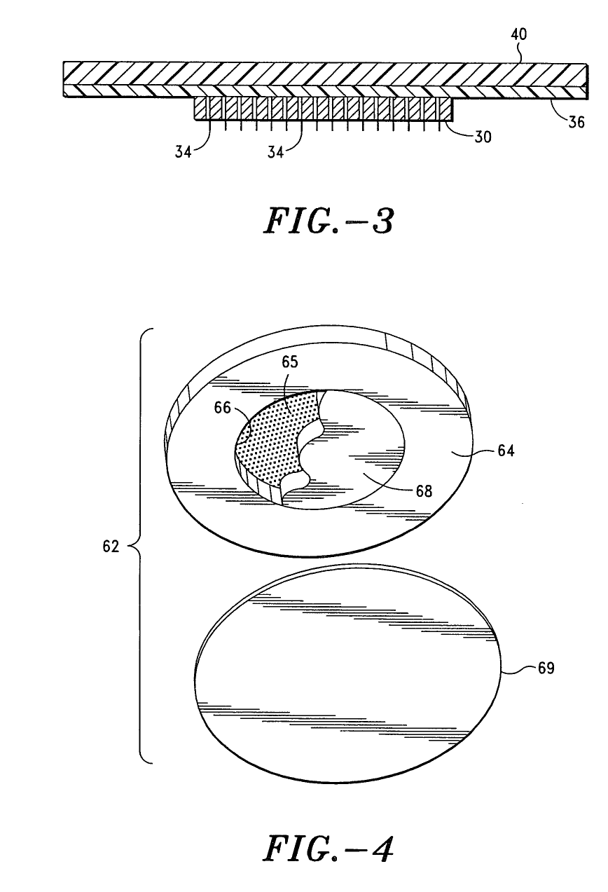

Referring now to FIG. 2, there is shown a microprojection member 31 having microprojections 34 that include a biocompatible coating 35. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 31 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

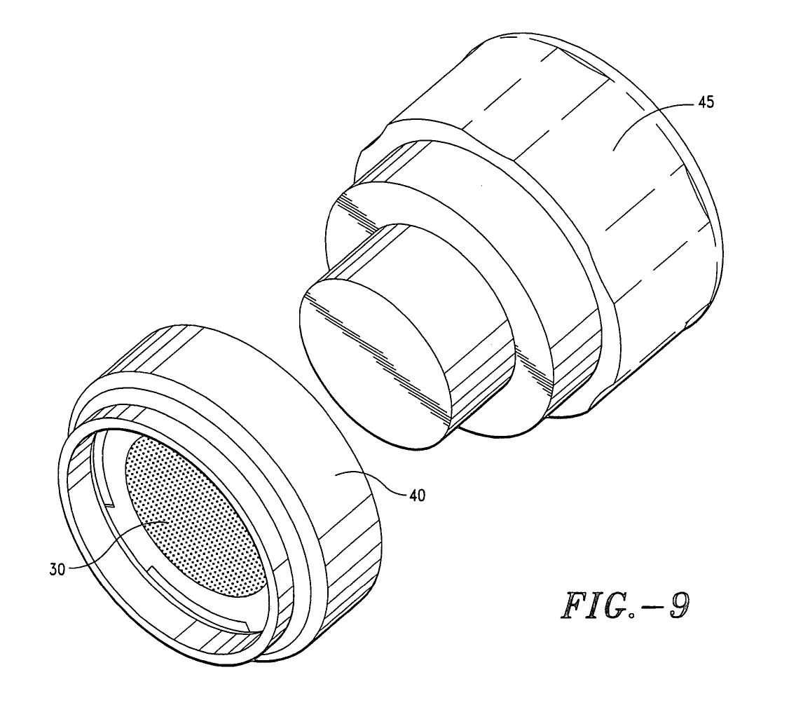

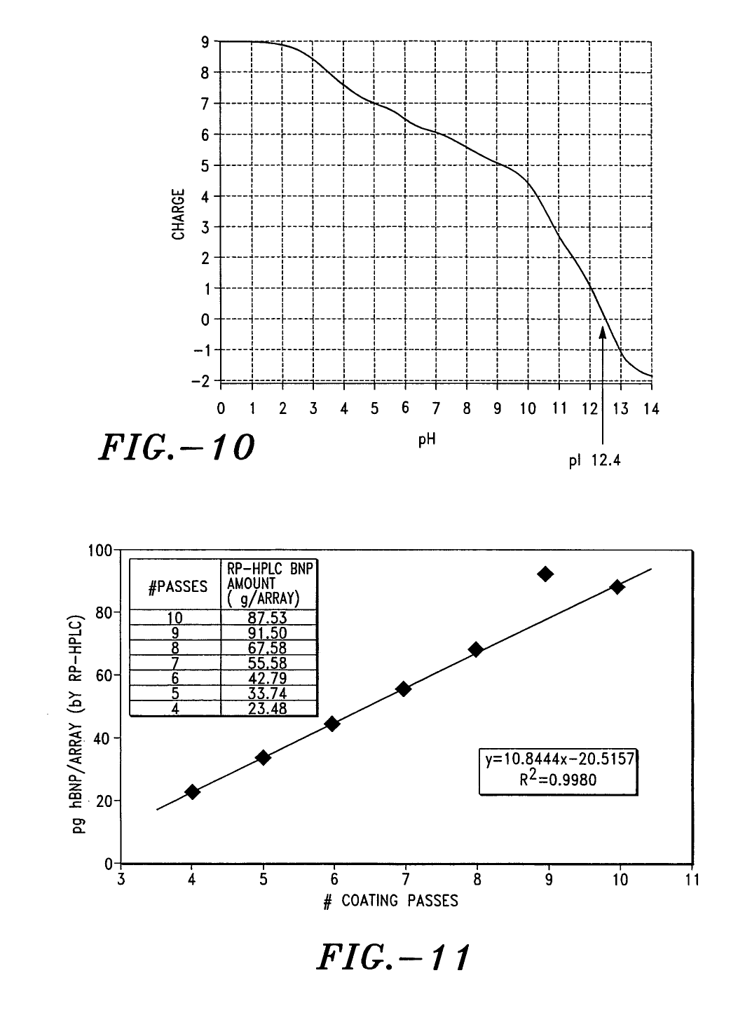

Referring now to FIGS. 7 and 8, for storage and application, the microprojection member (e.g., 30 or 31) is preferably suspended in a retainer ring 40 by adhesive tabs 6, as described in detail in U.S. application Ser. No. 09/976,762 (Pub. No. 2002/0091357), which is incorporated by reference herein in its entirety.

After placement of the microprojection member in the retainer ring 40, the microprojection member is applied to the patient's skin. Preferably, the microprojection member is applied to the patient's skin using an impact applicator 45, such as shown in FIG. 8 and described in Co-Pending U.S. application Ser. No. 09/976,978, which is incorporated by reference herein in its entirety.

As indicated, according to one embodiment of the invention, the coating formulations applied to the microprojection member 30 to form solid biocompatible coatings can comprise aqueous and non-aqueous formulations having at least one natriuretic peptide. According to the invention, the natriuretic peptide can be dissolved within a biocompatible carrier or suspended within the carrier.

In a preferred embodiment, the brain natriuretic peptide comprises a human B-type natriuretic peptide (BNP), including hBNP(1-32) and analogs, salts, variants, active fragments and simple derivatives thereof.

In one embodiment of the invention, the natriuretic peptide comprises in the range of approximately 1-30 wt. % of the coating formulation.

In one embodiment, the amount of the natriuretic peptide contained in the coating formulation is preferably in the range of approximately 1-2000 μg.

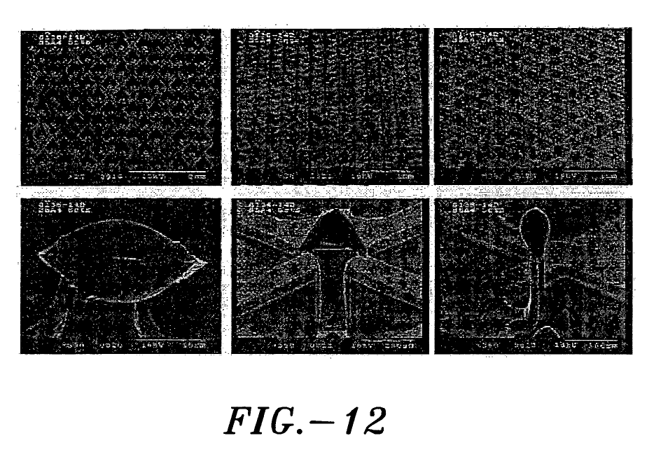

Referring now to FIG. 10, there is shown the predicted charge profile of hBNP(1-32), a peptide presenting four basic pKa (Arg, Lys, Cys, and Tyr), and three acidic pKa (His, Asp, and Glu). As illustrated in FIG. 10, at pH 11.5 the peptide presents a zero net electric charge. This point is also called the isoelectric point or pI. Since the pI of hBNP (1-32) is so high, it is anticipated that the neutral species mostly exists at a pH >8. In this pH range, the peptide is expected to precipitate out of solution.

Accordingly, in a preferred embodiment, the pH of the coating formulation is below approximate pH 9. More preferably, the pH of the coating formulation is in the range of approximately pH 3-pH 8. Even more preferably, the pH of the coating formulation is in the range of approximately pH 4-pH 6.

In one embodiment of the invention, the coating formulation includes at least one of the aforementioned buffers.

In one embodiment of the invention, the coating formulation includes at least one surfactant. According to the invention, the surfactant(s) can be zwitterionic, amphoteric, cationic, anionic, or nonionic. Examples of suitable surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80 and SDS.

In one embodiment of the invention, the concentration of the surfactant is in the range of approximately 0.001-2 wt. % of the coating formulation.

In a further embodiment of the invention, the coating formulation includes at least one polymeric material or polymer that has amphiphilic properties. Examples of the noted polymers include, without limitation, cellulose derivatives, such as hydroxyethylcellulose (HEC), hydroxypropylmethylcellulose (HPMC), hydroxyl-propycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation. Even more preferably, the concentration of the polymer is in the range of approximately 0.1-5 wt. % of the coating formulation.

According to the invention, the coating formulation can further include a hydrophilic polymer. Preferably, the hydrophilic polymer is selected from the following group: hydroxyethyl starch, dextran, poly(vinyl alcohol), poly(ethylene oxide), poly(2-hydroxyethylmethacrylate), poly(n-vinyl pyrolidone), polyethylene glycol and mixtures thereof, and like polymers. As is well known in the art, the noted polymers increase viscosity.

The concentration of the hydrophilic polymer in the coating formulation is preferably in the range of approximately 0.01-20 wt. %, more preferably, in the range of approximately 0.03-10 wt. % of the coating formulation.

According to the invention, the coating formulation can further include a biocompatible carrier, such as those disclosed in Co-Pending U.S. application Ser. No. 10/127,108, which is incorporated by reference herein in its entirety. Examples of suitable biocompatible carriers include, without limitation, human albumin, bioengineered human albumin, polyglutamic acid, polyaspartic acid, polyhistidine, pentosan polysulfate, polyamino acids, sucrose, trehalose, melezitose, raffinose and stachyose.

The concentration of the biocompatible carrier in the coating formulation is preferably in the range of approximately 2-70 wt. %, more preferably, in the range of approximately 5-50 wt. % of the coating formulation.

In a further embodiment, the coating formulation includes at least one stabilizing agent, which can comprise, without limitation, a non-reducing sugar, a polysaccharide or a reducing sugar.

Suitable non-reducing sugars include, for example, sucrose, trehalose, stachyose, or raffinose.

Suitable polysaccharides include, for example, dextran, soluble starch, dextrin, and insulin.

Suitable reducing sugars include, for example, monosaccharides, such as apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides, such as primeverose, vicianose, rutinose, scillabiose, cellobiose, gentiobiose, lactose, lactulose, maltose, melibiose, sophorose, and turanose, and the like.

The coating formulations and, hence, biocompatible coatings of the invention can further include a vasoconstrictor, such as those disclosed in Co-Pending U.S. application Ser. No. 10/674,626, which is incorporated by reference herein in its entirety. As set forth in the noted Co-Pending Application, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

As will be appreciated by one having ordinary skill in the art, the addition of a vasoconstrictor to the coating formulations and, hence, solid biocompatible coatings of the invention (or the hydrogel formulations or solid state formulation, discussed below) is particularly useful to prevent bleeding that can occur following application of the microprojection member or array and to prolong the pharmacokinetics of the natriuretic peptide through reduction of the blood flow at the application site and reduction of the absorption rate from the skin site into the system circulation.

The concentration of the vasoconstrictor, if employed, is preferably in the range of approximately 0.1 wt. % to 10 wt. % of the coating formulation.

In yet another embodiment ofthe invention, the coating formulation includes at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride) and zwitterionic compounds (e.g., amino acids).

The term “pathway patency modulator”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent which can comprise Alpha-Cyclodextrin, Beta-Cyclodextrin, Gamma-Cyclodextrin, glucosyl-alpha-Cyclodextrin, maltosyl-alpha-Cyclodextrin, 2-hydroxypropyl-beta-Cyclodextrin, 2-hydroxypropyl-gamma-Cyclo-dextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. The most preferred solubilising/complexing agents comprise beta-cyclodextrin, hydroxypropyl beta-cyclodextrin, 2-hydroxypropyl-beta-Cyclodextrin and sulfobutylether7 beta-cyclodextrin.

The concentration of the solubilising/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the concentration of the non-aqueous solvent is in the range of approximately 1 wt. % to 50 wt. % of the coating formulation.

Other known formulation adjuvants can also be added to the coating formulations; provided, they do not adversely affect the necessary solubility and viscosity characteristics of the coating formulation and the physical integrity of the dried coating.

Preferably, the coating formulations have a viscosity less than approximately 500 centipoise and greater than 3 centipose.

In one embodiment of the invention, the coating thickness is less than 25 microns, more preferably, less than 10 microns as measured from the microprojection surface.

The desired coating thickness is dependent upon several factors, including the required dosage and, hence, coating thickness necessary to deliver the dosage, the density of the microprojections per unit area of the sheet, the viscosity and concentration of the coating composition and the coating method chosen.

According to the invention, after a coating formulation has been applied to the microprojections 34, the coating formulation can be dried by various means. In a preferred embodiment of the invention, the coated microprojection member 30 is dried in ambient room conditions. However, various temperatures and humidity levels can be used to dry the coating formulation onto the microprojections. Additionally, the coated member can be heated, stored under vacuum or over desiccant, lyophilized, freeze dried or similar techniques used to remove the residual water from the coating.

Referring now to FIG. 6, there is shown a further microprojection (or delivery) system (designated generally “80”) that can be employed within the scope of the present invention. As illustrated in FIG. 6, the system 80 includes a gel pack 62 and a microprojection assembly 70, having a microprojection member, such as the microprojection member 30 shown in FIG. 1.

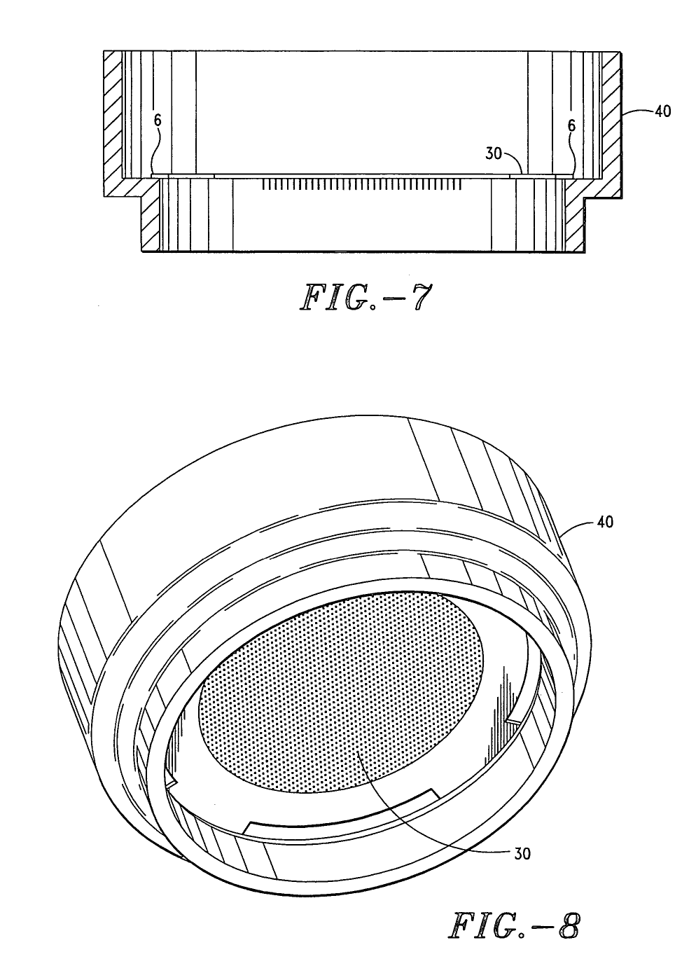

Referring now to FIG. 5, the microprojection assembly 70 includes a backing membrane ring 72 and a similar microprojection array 32. The microprojection assembly further includes a skin adhesive ring 74.

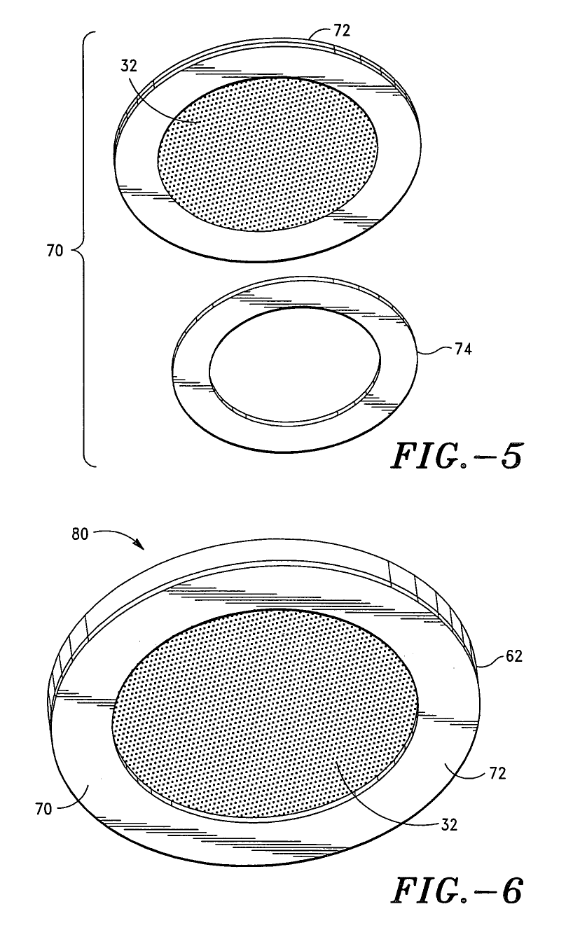

Referring now to FIG. 4, the gel pack 62 includes a housing or ring 64 having a centrally disposed reservoir or opening 66 that is adapted to receive a predetermined amount of a hydrogel formulation 68 therein. As illustrated in FIG. 4, the ring 64 further includes a backing member 65 that is disposed on the outer planar surface of the ring 64. Preferably, the backing member 65 is impermeable to the hydrogel formulation.

In a preferred embodiment, the gel pack 62 further includes a strippable release liner 69 that is adhered to the outer surface of the gel pack ring 64 via a conventional adhesive. As described in detail below, the release liner 69 is removed prior to application of the gel pack 62 to the applied (or engaged) microprojection assembly 70.

Further details of the illustrated gel pack 62 and microprojection assembly 70, as well as additional embodiments thereof that can be employed within the scope of the present invention are set forth in Co-Pending application Ser. No. 10/971,430, which is incorporated by reference herein in its entirety.

As indicated above, in at least one embodiment of the invention, the hydrogel formulation contains at least one natriuretic peptide. In an alternative embodiment of the invention, the hydrogel formulation is devoid of a natriuretic peptide and, hence, is merely a hydration mechanism.

According to the invention, when the hydrogel formulation is devoid of a natriuretic peptide, the natriuretic peptide is either disposed in a coating on the microprojection array 32, as described above, or contained in a solid state formulation, such as disclosed in PCT Pub. No. WO 98/28037, which is similarly incorporated by reference herein in its entirety, on the skin side of the microprojection array 32, such as disclosed in the noted Co-Pending application Ser. No. 10/971,430 or the top surface of the array 32.

Preferably, the hydrogel formulations of the invention comprise water-based hydrogels. Hydrogels are preferred formulations because of their high water content and biocompatibility.

As is well known in the art, hydrogels are macromolecular polymeric networks that are swollen in water. Examples of suitable polymeric networks include, without limitation, dextran, hydroxyethyl starch, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics. The most preferred polymeric materials are cellulose derivatives. These polymers can be obtained in various grades presenting different average molecular weight and therefore exhibit different rheological properties.

Preferably, the concentration of the polymeric material is in the range of approximately 0.5-40 wt. % of the hydrogel formulation.

The hydrogel formulations of the invention preferably have sufficient surface activity to insure that the formulations exhibit adequate wetting characteristics, which are important for establishing optimum contact between the formulation and the microprojection array and skin and, optionally, the solid state formulation.

According to the invention, adequate wetting properties are achieved by incorporating a wetting agent, such as a surfactant or polymeric material having amphiphilic properties, in the hydrogel formulation. Optionally, a wetting agent can also be incorporated in the solid state formulation.

According to the invention, the surfactant(s) can be zwitterionic, amphoteric, cationic, anionic, or nonionic. Examples of suitable surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laureate, and alkoxylated alcohols such as laureth-4. Most preferred surfactants include Tween 20, Tween 80 and SDS.

Examples of suitable polymers include, without limitation, cellulose derivatives, such as hydroxyethyl starch, hydroxyethylcellulose (HEC), hydroxypropyl-methylcellulose (HPMC), hydroxypropycellulose (HPC), methylcellulose (MC), hydroxyethylmethylcellulose (HEMC), or ethylhydroxyethylcellulose (EHEC), as well as pluronics and dextran.

Preferably, the concentration of the surfactant is in the range of approximately 0.001-2 wt. % of the hydrogel formulation. The concentration of the polymer that exhibits amphiphilic properties is preferably in the range of approximately 0.01-20 wt. % of the hydrogel formulation.

As will be appreciated by one having ordinary skill in the art, the noted wetting agents can be used separately or in combinations.

In a further embodiment of the invention, the hydrogel formulation includes a solubilizing/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl-beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred are beta-cyclodextrin, hydroxypropyl-beta-Cyclodextrin, 2-hydroxypropyl-beta-Cyclodextrin and sulfobutylether7 beta-cyclodextrin.

In another embodiment of the invention, the hydrogel formulation includes at least one non-aqueous solvent, such as ethanol, isopropanol, methanol, propanol, butanol, propylene glycol, dimethyl sulphoxide and polyethylene glycol 400. Preferably, the concentration of the non-aqueous solvent is in the range of approximately 1 wt. % to 50 wt. % of the hydrogel formulation.

According to the invention, the hydrogel formulations can similarly include at least one pathway patency modulator, such as those disclosed in Co-Pending U.S. application Ser. No. 09/950,436. As indicated above, the pathway patentcy modulator can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextran sulfate sodium and EDTA.

The hydrogel formulation can further include at least one vasoconstrictor. Suitable vasoconstrictors include, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

The hydrogel formulations of the invention exhibit adequate viscosity so that the formulation can be contained in the gel pack 62, keeps its integrity during the application process, and is fluid enough so that it can flow through the microprojection assembly openings and into the skin pathways.

For hydrogel formulations that exhibit Newtonian properties, the viscosity of the hydrogel formulation is preferably in the range of approximately 2-300 poise (P), as measured at 25° C. For shear-thinning hydrogel formulations, the viscosity, as measured at 25° C., is preferably in the range of 1.5-30 P or 0.5 and 10 P, at shear rates of 667/s and 2667/s, respectively. For dilatant formulations, the viscosity, as measured at 25° C., is preferably in the range of approximately 1.5-30 P, at a shear rate of 667/s.

As indicated, in at least one embodiment of the invention, the hydrogel formulation contains at least one natriuretic peptide. According to the invention, when the hydrogel formulation contains a natriuretic peptide, the natriuretic peptide can be present at a concentration in excess of saturation or below saturation.

In one embodiment of the invention, the concentration of the natriuretic peptide is preferably in the range of at least 0.1-2 wt. % of the hydrogel formulation.

Preferably, the dose of natriuretic peptide delivered intracutaneously is in the range of approximately 10-2000 μg/day, more preferably, approximately 10-1000 μg/day.

In accordance with yet another embodiment of the invention, the microprojection system for delivering a natriuretic peptide comprises (i) a microprojection member having top and bottom surfaces, a plurality of openings that extend through the microprojection member and a plurality of microprojections that project from the bottom surface of the microprojection member, (ii) a gel pack containing a hydrogel formulation, and (iii) a solid state formulation having at least one natriuretic peptide. Details of the noted system are set forth in Co-Pending application Ser. No. 60/514,433, which is incorporated by reference herein in its entirety.

In accordance with one embodiment of the invention, the solid state formulation is disposed proximate the top surface of the microprojection member. In another embodiment, the solid state formulation is disposed proximate the bottom surface of the microprojection member.

In a preferred embodiment, the hydrogel formulation is devoid of a natriuretic peptide and thus functions as a hydration medium.

In one embodiment, the solid state formulation is a solid film made by casting a liquid formulation comprising at least one natriuretic peptide, a polymeric material, such as hyroxyethyl starch, dextran, hydroxyethylcellulose (HEC), hydroxypropylmethylcellulose (HPMC), hydroxypropycellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone) and pluronics, a plasticizing agent, such as glycerol, propylene glycol and polyethylene glycol, a surfactant, such as Tween 20 and Tween 80, and a volatile solvent, such as water, isopropanol, methanol and ethanol.

In one embodiment, the liquid formulation used to produce the solid film comprises: 0.1-20 wt. % natriuretic peptide, 5-40 wt. % polymer, 5-40 wt. % plasticizer, 0-2 wt. % surfactant, and the balance of volatile solvent.

Following casting and subsequent evaporation of the solvent, a solid film is produced.

Preferably, the natriuretic peptide is present in the liquid formulation used to produce the solid film at a concentration in the range of approximately 0.1-2 wt. %.

In another embodiment of the invention, the solid state formulation is a powder or cake formulation. Suitable formulations are achieved by spray drying, freeze drying, spray freeze drying and supercritical fluid processing. According to the invention, these methods form a high payload powder or cake solid state formulation that is reconstituted by the hydrogel formulation prior to the transdermal delivery of the natriuretic peptide. Preferably, the powder formulations are adapted to have relatively high porosity to facilitate reconstitution and improve patient compliance.

The noted processes of making powder and cake formulations are highly efficient, typically having yields of approximately 85%. Further, the processes do not require the use of plasticizers that depress Tg and, correspondingly, can reduce shelf life. Preferably, the formulations subjected to drying or supercritical fluid extraction in the noted methods also comprise a carbohydrate, such as a saccharide or a sugar alcohol to help protect the natriuretic peptide. Also preferably, the formulation includes an antioxidant, such as methionine. Specific formulations are discussed below.

Spray drying, freeze drying, spray freeze drying and supercritical fluid extraction afford good control over particle size and distribution, particle shape and morphology. The noted techniques are also known in the art. For example, the spray freeze drying process is ideal for high valued therapeutic drugs as batch sizes as small as 300 mg can be produced with high yields.

As can be appreciated, the spray drying, freeze drying, spray freeze drying and supercritical fluid extraction processes generate a cake form which is readily incorporated into the microprojection system discussed above. Alternatively, the processes generate a powder form, which is further processed to form a cake. In other embodiments, the powder form is held in a container adapted to communicate with the hydrogel. Preferably, such embodiments include stripable release liners to separate the powder form from the hydrogel until reconstitution is desired.

In one embodiment of the invention, a suitable spray freeze drying process generally involves exposing an atomized liquid formulation containing the natriuretic peptide to liquid nitrogen. Under the reduced temperature, the atomized droplets freeze in a time-scale of milliseconds. This freezing process generates very fine ice crystals, which are subsequently lyophilized. The noted technique generates a powder having a high intraparticle porosity, allowing rapid reconstitution in aqueous media. Examples of suitable nesiritide formulations are given below.

In another embodiment of the invention, a suitable supercritical fluid process generally involves crystallizing a liquid formulation of the natriuretic peptide in a solvent that is maintained above its critical temperature and pressure. Controlling the conditions of the crystallization process allows the production of a natriuretic peptide powder having desired particle size and distribution, particle shape and morphology.

Preferably, the pH of the liquid formulation used to produce the solid state formulation is below about pH 6. More preferably, the pH of the formulation used to produce the solid state formulation is in the range of approximately pH 3-pH 6. Even more preferably, the pH of the liquid formulation used to produce the solid state formulation is in the range of approximately pH 4-pH 6.

In another embodiment, the liquid formulation used to produce the solid state formulation includes a stabilizing agent, which can comprise, without limitation, a non-reducing sugar, a polysaccharide or a reducing sugar.

Suitable non-reducing sugars include, for example, sucrose, trehalose, stachyose, or raffinose.

Suitable polysaccharides include, for example, dextran, soluble starch, dextrin, and insulin.

Suitable reducing sugars include, for example, monosaccharides such as apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as primeverose, vicianose, rutinose, scillabiose, cellobiose, gentiobiose, lactose, lactulose, maltose, melibiose, sophorose, and turanose, and the like.

In one embodiment of the invention, the liquid formulation used to produce the solid state formulation includes at least one of the aforementioned buffers.

In another embodiment of the invention, the liquid formulation used to produce the solid state formulation includes at least one of the aforementioned complexing/solubilizing agents.

In a further embodiment of the invention, the liquid formulation used to produce the solid state formulation includes at least one of the aforementioned vasoconstrictors.

In accordance with one embodiment of the invention, the method for delivering a natriuretic peptide to patient comprises the following steps: (i) providing a microprojection member 31 having a plurality of microprojections 34, the microprojection member 31 including a biocompatible coating having at least one natriuretic peptide disposed therein, and (ii) applying the coated microprojection member 31 to the patient's skin via an actuator, whereby the microprojections 34 pierce the stratum corneum to achieve local or systemic therapy.

The coated microprojection member 31 is preferably left on the skin for a period lasting from 5 seconds to 24 hours. Following the desired wearing time, the microprojection member 31 is removed from the patient's skin.

In accordance with a further embodiment of the invention, the method for delivering a natriuretic peptide to a patient comprises the following steps: (i) providing a microprojection assembly 70 having a microprojection member 30 and a gel pack 62, the microprojection member 30 including a plurality of microprojections 34, the gel pack 62 including a hydrogel formulation 68 having at least one natriuretic peptide, (ii) applying the microprojection member 30 to the patient's skin, whereby the microprojections 34 pierce the patient's stratum corneum and form a plurality of microslits therein, (iii) removing the release liner 69 from the gel pack 62 (if employed), (iv) and placing the gel pack 60 on the microprojection member 30, whereby the hydrogel formulation 68 is released from the gel pack 62 and migrates through the openings 38 in the microprojection array 32, down the outer surfaces of the microprojections 34 and into and through the microslits formed by the microprojections 34 to achieve local or systemic therapy.

Preferably, the gel pack 62 is left on the patient's skin for a period in the range of approximately 5 min. to 24 hours. Following the desired wearing time, the gel pack 62 and microprojection member 30 are removed from the skin.

In one embodiment of the invention, the microprojection assembly 70 includes a biocompatible coating having at least one natriuretic peptide, which is disposed on the microprojection member 31, more preferably, the microprojections 34.

In a further embodiment, at least one natriuretic peptide is contained in both the hydrogel formulation 68 and the biocompatible coating disposed on the microprojection member 31.

According to a further embodiment of the invention, the microprojection member 30 is applied to the patient's skin and removed. The release liner 69 (if employed) is then removed from the gel pack 62 and the gel pack 62 is placed on the pretreated skin, whereby the hydrogel formulation 68 having at least one natriuretic peptide is released from the gel pack 62 and passes through the microslits in the stratum corneum formed by the microprojections 34 to achieve local or systemic therapy.

Preferably, the gel pack 62 is left on the patient's skin for a period in the range of approximately 5 min. to 24 hours. Following the desired wearing time, the gel pack 62 is removed from the skin.

In accordance with another embodiment of the invention, the method for delivering a natriuretic peptide to a patient comprises the following steps: (i) providing a microprojection assembly 70 having a microprojection member 30, a gel pack 62 and a solid state formulation disposed proximate to (or on) the microprojection member 30, the microprojection member 30 including a plurality of microprojections 34, the gel pack 62 including a hydrogel formulation 68 and the solid state formulation including at least one natriuretic peptide, (ii) applying the microprojection member 30 to the patient's skin, whereby the microprojections 34 pierce the patient's stratum corneum and form a plurality of microslits therein, (iii) removing the release liner 69 from the gel pack 62 (if employed), and (iv) placing the gel pack 60 on the microprojection member 30, whereby the hydrogel formulation 68 is released from the gel pack 62 and migrates through the solid state formulation and the openings 38 in the microprojection array 32, down the outer surfaces of the microprojections 34 and into and through the microslits formed by the microprojections 34 to achieve local or systemic therapy.

Preferably, the gel pack 62 is left on the patient's skin for a period in the range of approximately 5 min to 24 hours. Following the desired wearing time, the gel pack 62 and microprojection member 30 are removed from the skin.

Preferably, the dose of natriuretic peptide delivered intracutaneously (per day), in accordance with each of the noted embodiments, is in the range of approximately 10-2000 μg/day, more preferably, approximately 10-1000 μg/day.

According to the invention, the noted dosage can be administered in various regimes. By way of example, the noted dosage can be administered once or twice weekly for 12-26 weeks or 12-24 days for 12 weeks.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169,383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by attained in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods, such as sonophoresis or piezoelectric devices, can be used in conjunction with the invention.

When the invention is employed in conjunction with electrotransport, sonophoresis or piezoelectric systems, the microprojection assembly 70 is first applied to the skin as explained above. The release liner 69 is removed from the gel pack 62, which is part of the electrotransport, sonophoresis or piezoelectric system. This assembly is then placed on the skin template, whereby the hydrogel formulation 68 is released from the gel pack 62 and passes through the microslits in the stratum corneum formed by the microprojections 34 to achieve local or systemic therapy with additional facilitation of drug transport via the electrotransport, sonophoresis or piezoelectric processes. When the invention is employed in conjunction with one of the noted systems, the total skin contact area can be in the range of approximately 2-120 cm².

### EXAMPLES

The following examples are given to enable those skilled in the art to more clearly understand and practice the present invention. They should not be considered as limiting the scope of the invention but merely as being illustrated as representative thereof.

### Example 1

Coating Feasibility

The coating feasibility of coating a simple sucrose formulation (i.e., 20% hBNP, 20% sucrose, 0.05% polysorbates 20) was evaluated in a pilot plant facility on a coater having a coating reservoir fitted with a 0.621 in. drum, which provided a doctor blade gap of approximately 100 μm. The coater was placed in a dehumidified laminar air-flow hood (LAF) set to maintain a dew point of approximately 1° C. The film temperature was maintained to 0.5-1° C. above the dew point by circulating a chilled fluid through a heat transfer block mounted below the reservoir. The coolant was chilled to −3.2° C.

For coating feasibility, 500 μL of a 20% hBNP, 20% sucrose, 0.05% polysorbates solution was added to the reservoir and the drum speed was increased to 50 RPM. Strips were passed over the film at a coating height of 250 μm. Strips were coated with various passes ranging from 4 10 to determine the level and linearity of the coated amount. A sample of the coating solution was removed from the reservoir after one hour to evaluate the stability of the peptide under sustained applied shear stress of the coater.

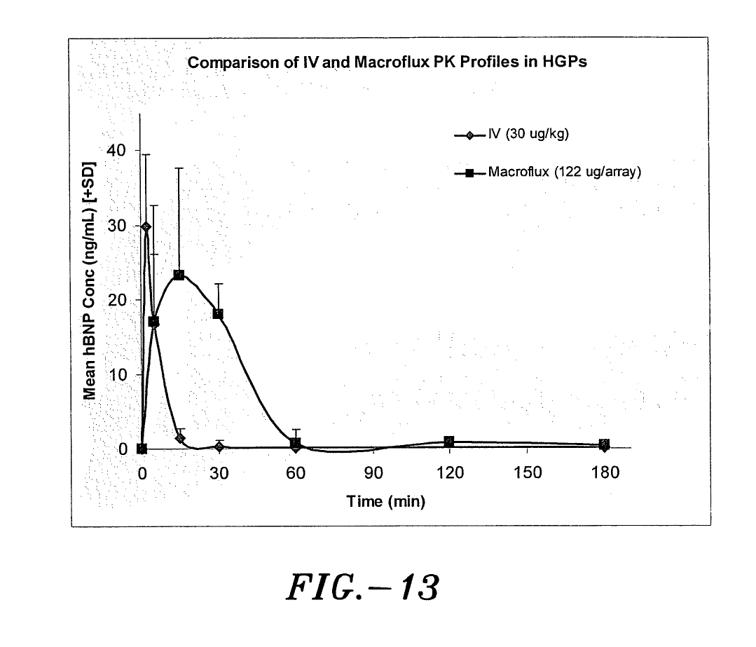

Samples of the coated arrays at each level were analyzed by RP-HPLC following extraction from the microprojection tips by dissolution in water. The results of the analyses are set forth in FIG. 11.

The coating solution was also analyzed by RP-HPLC before and after the coating experiment and was stored at 2-8° C. over night to determine the solution stability. The results of this study are set forth in Table I.

TABLE I

| | | Post Coating | Post Coating |
|---|---|---|---|
| Sample | Pre-Coating | Initial | 24 hour @5° C. |
| RP-HPCL | 200.47 | 202.64 | 203.30 |
| BNP amount (μg/array) | | | |
| BNP | 97.78 | 97.81 | 97.91 |
| (% purity) | | | |
| Impurity @ RRT: 0.44 | 0.19 | 0.16 | 0.10 |
| (%) | | | |
| Impurity @ RRT: 0.60 | 0.29 | 0.26 | 0.21 |
| (%) | | | |
| Impurity @ RRT: 0.81 | 0.28 | 0.29 | 0.30 |
| (%) | | | |
| Impurity @ RRT: 0.84 | 0.17 | 0.17 | 0.17 |
| (%) | | | |
| Impurity @ RRT: 0.92 | 0.47 | 0.46 | 0.46 |
| (%) | | | |
| Impurity @ RRT: 0.96 | 0.26 | 0.27 | 0.27 |
| (%) | | | |
| Impurity @ RRT: 1.06 | 0.42 | 0.42 | 0.41 |
| (%) | | | |
| Impurity @ RRT: 1.27 | 0.14 | 0.16 | 0.16 |
| (%) | | | |

As reflected in Table I, the coating solution showed good stability throughout the coating study and did not show any increased degradation under sustained shear stress for one hour in the coating reservoir.

A sample of the noted post coating solution was also analyzed under an optical microscope. No evidence of fibril formation was detected.

Morphology of Coated Arrays

Arrays coated with the 20% hBNP, 20% sucrose, 0.05% polysorbates 20 formulation were analyzed under scanning electron microscopy (SEM, Hitachi S-2460N emission current 60 μA, acceleration voltage 16 kV). The images of samples coated with 10, 8 and 6 passes are shown in FIG. 12 and identified as A, B and C, respectively.

As illustrated in FIG. 12, the images reflect good tip coating morphology.

### Example 2

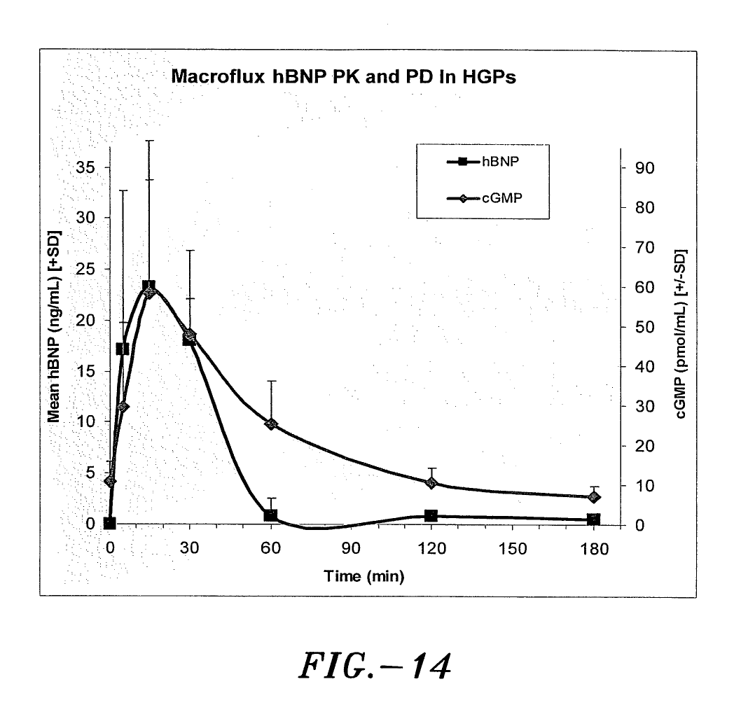

The following example demonstrates the pharmacokinetic and pharmacodynamic responses in male HGPs after transdermal, intraveneous (IV) and subcutaneous injection of hBNP. Referring first to FIG. 13, there is shown the pharmacokinetic response in male HGPs receiving hBNP administered by intravenous (IV) route (closed diamonds) and transdermal delivery using microprojections dry-coated with drug (closed squares). For IV administration, the hBNP was prepared in phosphate buffered saline and injected into animals at 30 μh hBNP/kg. Plasma hBNP levels were determined at t=0, 2, 15, 30, 60, and 180 min. post injection. For transdermal adminstration, the hBNP (31.65% [w/w]) was formulated with sucrose (6.25%[w/w]), polysorbate 20 (6=0.10%[w/w]), and USP water for injection (62%) then coated onto microprojection arrays (2 cm²), forming a thin-dry film (112 μg hBNP/array).

The microprojection arrays were applied on HGPs (˜149 μg hBNP/kg) for 60 minutes then removed. Plasma hBNP levels were determined at t=0, 5, 15, 30, 60, and 180 min. after microprojection application. The results shown in FIG. 13 represent average hBNP levels (n=5 HGP/group) measured by immunoassay

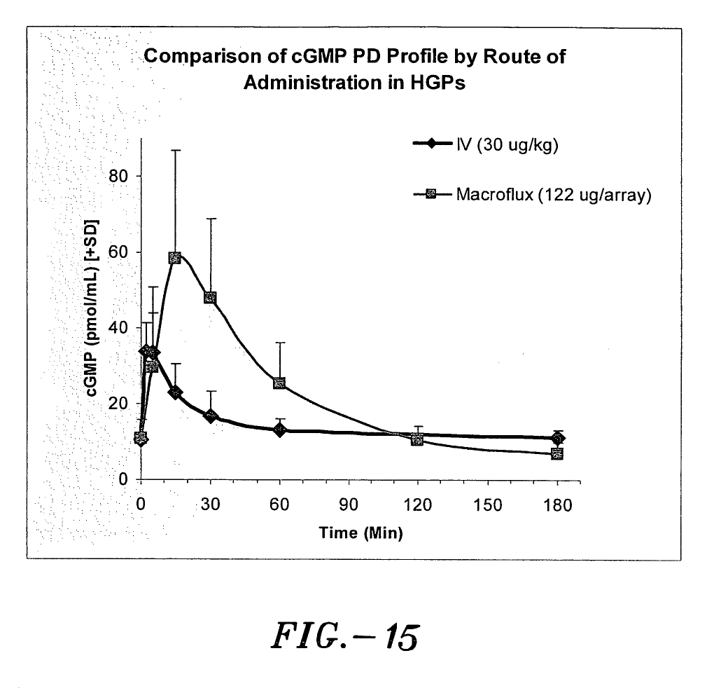

Referring now to FIG. 14, there is shown the pharmacokinetic (closed squares) and pharmacodynamic (closed diamonds) response in HGP receiving hBNP by transdermal delivery using coated microprojections. The hBNP was formulated as described above.

The microprojection arrays were applied on HGPs (˜149 μg hBNP/kg) for 60 minutes then removed. Plasma was collected at t=0, 5, 15, 30, 60, and 180 min. after microprojection application and measured for hBNP and cGMP by immunoassay. The results shown in FIG. 14 represent average hBNP and cGMP levels (n=5 HGP).

Referring now to FIG. 15, there is shown a comparison of the pharmacodynamic response between IV administration and transdermal delivery of hBNP using microprojections. Administration of the hBNP by IV route and by microprojection arrays, as well as plasma collection, was performed as described above.

The results shown in FIG. 15 represent average cGMP levels (n=5 HGP/group) measured by immunoassay.

### Example 3

Five hBNP solid state formulations were prepared by freeze drying and spray freeze drying processes to assess reconstitution time. In each case the reconstitution medium was deionised water and the amount added to each formulation was such that the resulting concentration of hBNP was 100 mg/ml. The hBNP spray freeze dried powder or freeze dried cake was allowed to dissolve without the aid of agitation after addition of deionised water to the powder hBNP formulations. The reconstitution results are shown in Table II.

TABLE II

| | | | Reconstitution | State after |
|---|---|---|---|---|
| Lot No. | Composition | Process | time (min) | reconstitution |
| 8269166A | 49% w/w hBNP, | SFD | 1 | Liquid |
| | 49% w/w sucrose, | | | |
| | 2% methionine | | | |
| | (50% solids | | | |
| | content). | | | |
| 8269166B | 49% w/w hBNP, | SFD | 1 | Liquid |
| | 49% w/w sucrose, | | | |
| | 2% methionine | | | |
| | (30% solids | | | |
| | content). | | | |
| 8269170A | 5.1% w/w hBNP, | FD | 1.5 | Liquid |
| | 5.1% w/w | | | |
| | sucrose, 1.3% | | | |
| | w/w mannitol, | | | |
| | 0.2% w/w | | | |
| | methionine. | | | |
| 8269170B | 5.0% w/w hBNP, | FD | 1.5 | Liquid |
| | 5.0% w/w | | | |
| | sucrose, 2.5% | | | |
| | w/w mannitol, | | | |
| | 0.2% w/w | | | |
| | methionine. | | | |
| 8269170C | 5.1% w/w hBNP, | FD | 1.5 | Liquid |
| | 2.6% w/w | | | |
| | sucrose, 2.6% | | | |
| | w/w mannitol, | | | |
| | 0.2% w/w | | | |
| | methionine. | | | |

### Example 4

In this example, the storage stability of powder and cake solid state formulations was assessed. Three formulations were prepared and dispensed in glass vials under an inert atmosphere. The glass vials were capped and stored at ambient temperature and 40° C. for a period of two weeks to determine stability. As shown in Table III, the freeze dried and spray freeze dried formulations exhibit adequate stability over the storage period.

TABLE III

| | | Formulation | T = 0 hBNP | T = 2 weeks |
|---|---|---|---|---|
| Process | Lot No | composition | Purity (%) | hBNP Purity (%) |
| FD | 8520005A | 43.5% w/w | 98.16 | 25° C. - 97.85 |
| | | hBNP, 43.5% | | 40° C. - 96.85 |
| | | w/w sucrose, | | |
| | | 10.9% w/w | | |
| | | mannitol, 2.0% | | |
| | | w/w methionine | | |
| | 8520005A | 49.0% w/w | 97.93 | 25° C. - 97.85 |
| | | hBNP, 49.0% | | 40° C. - 97.23 |
| | | w/w sucrose, | | |
| | | 2.0% w/w | | |
| | | methionine | | |
| SFD | 8520007 | 49% w/w | 97.87 | 25° C. - 97.71 |
| | | hBNP, 49% w/w | | 40° C. - 96.95 |
| | | sucrose, 2% w/w | | |
| | | methionine (40% | | |
| | | solids content) | | |

As will be appreciated by one having ordinary skill in the art, the present invention provides numerous advantages. Among the advantages are the provision of apparatus and methods for intracutaneous administration of natriuretic peptides with improved pharmacokinetics, including rapid on-set with tolerable C_{MAX} and biological action of the natriuretic peptide(s) for a period of 2-6 hours.

A further advantage of the present invention is that the formulations employed as and to produce the delivery mediums substantially inhibit oxidation of the natriuretic peptide(s) disposed therein. The stability of the agent-containing medium is thus significantly enhanced.

Additional advantages include a decreased risk of complications compared to parental injections and increased patient compliance by virtue of the convenience and tolerability associated with administration of the microprojection member (i.e., patch).

The apparatus and methods of the invention can also be employed in the treatment of various ailments, including, but not limited to, STEMI (ST-Segment Elevation Myocardial Infarction), CKD (Chronic Kidney Disease), acute coronary syndromes (Class III/IV heart failure), pulmonary hypertension and pre-eclampsia.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A delivery system for transdermally delivering a natriuretic peptide to a patient, comprising:
a microprojection member having a plurality of microprojections that are adapted to pierce the stratum corneum of the patient; and
a biocompatible coating disposed on said microprojection member, said coating being formed from a coating formulation having at least one natriuretic peptide disposed therein.

2. The delivery system of claim 1, wherein said coating is disposed on at least one of said plurality of microprojections.

3. The delivery system of claim 1, wherein said coating formulation comprises an aqueous formulation.

4. The delivery system of claim 1, wherein said coating formulation comprises a non-aqueous formulation.

5. The delivery system of claim 1, wherein said natriuretic peptide is selected from the group consisting of artrial natriuretic peptides (ANP), B-type natriuretic peptides (BNP), C-type natriuretic peptides and urodilatins, and analogs, active fragments, degradation products, salts and simple derivatives and combinations thereof.

6. The delivery system of claim 5, wherein said natriuretic peptide comprises hBNP(1-32).

7. The delivery system of claim 1, wherein said natriuretic peptide comprises in the range of approximately 1-30 wt. % of said coating formulation.

8. The delivery system of claim 1, wherein said natriuretic peptide comprises in the range of 1 μg-2000 μg of said biocompatible coating.

9. The delivery system of claim 1, wherein the pH of said coating formulation is below approximately pH 9.

10. The delivery system of claim 1, wherein said coating formulation includes at least one buffer selected from the group consisting of ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarbally acid, malonic acid, adipic acid, citraconic acid, glutaratic acid, itaconic acid, mesaconic acid, citramalic acid, dimethylopropionic acid, tiglic acid, glyceric acid, methacrylic acid, isocrotonic acid, β-hydroxybutyric acid, crotonic acid, angelic acid, hydracrylic acid, aspartic acid, glutamic acid, glycine and mixtures thereof.

11. The delivery system of claim 1, wherein said coating formulation includes at least one surfactant selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, sorbitan derivatives, alkoxylated alcohols and mixtures thereof.

12. The delivery device of claim 1, wherein said coating formulation includes at least one polymeric material having amphiphilic properties.

13. The delivery system of claim 1, wherein said coating formulation includes a hydrophilic polymer selected from the following group consisting of hydroxyethyl starch, dextran, poly(vinyl alcohol), poly(ethylene oxide), poly(2-hydroxyethyl-methacrylate), poly(n-vinyl pyrolidone), polyethylene glycol and mixtures thereof.

14. The delivery system of claim 1, wherein said coating formulation includes a biocompatible carrier selected from the group consisting of human albumin, bioengineered human albumin, polyglutamic acid, polyaspartic acid, polyhistidine, pentosan polysulfate, polyamino acids, sucrose, trehalose, melezitose, raffinose, stachyose, mannitol and like sugar alcohols.

15. The delivery system of claim 1, wherein said coating formulation includes a stabilizing agent selected from the group consisting of a non-reducing sugar, a polysaccharide and a reducing sugar.

16. The delivery system of claim 1, wherein said coating formulation includes at least one vasoconstrictor selected from the group consisting of amidephrine, cafaminol, cyclopentaimine, deoxyepinephrine, epinephrine, felypressin, indanzoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline, and mixtures thereof.

17. The delivery system of claim 1, wherein said coating formulation includes at least one pathway patency modulator selected from the group consisting of osmotic agents, zwitterionic compounds, anti-inflammatory agents and anticoagulants.

18. The delivery system of claim 1, wherein said coating formulation includes a solubilising/complexing agent selected from the group consisting of Alpha-Cyclodextrin, Beta-Cyclodextrin, Gamma-Cyclodextrin, glucosyl-alpha-Cyclodextrin, maltosyl-alpha-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin.

19. The delivery system of claim 1, wherein said coating formulation has a viscosity in the range of approximately 3-500 centipose.

20. The delivery system of claim 1, wherein the thickness of said biocompatible coating is less than approximately 25 microns.

21. A delivery system for transdermally delivering a natriuretic peptide to a patient, comprising:
a microprojection member having a plurality of microprojections that are adapted to pierce the stratum corneum of the patient; and
a hydrogel formulation having at least one natriuretic peptide, said hydrogel formulation being in communication with said microprojection member.

22. The delivery system of claim 21, wherein said natriuretic peptide comprises in the range of approximately 0.1-2 wt. % of the hydrogel formulation.

23. The delivery system of claim 21, wherein said natriuretic peptide is selected from the group consisting of artrial natriuretic peptides (ANP), B-type natriuretic peptides (BNP), C-type natriuretic peptides and urodilatins, and analogs, active fragments, degradation products, salts and simple derivatives and combinations thereof.

24. The delivery system of claim 21, wherein said natriuretic peptide comprises hBNP(1-32).

25. The delivery system of claim 21, wherein the pH of said hydrogel formulation is below pH 6.

26. The delivery system of claim 21, wherein said hydrogel formulation comprises a water-based hydrogel having a macromolecular polymeric network.

27. The delivery system of claim 21, wherein said hydrogel formulation includes at least one surfactant, selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, sorbitan derivatives, and alkoxylated alcohols.

28. A delivery system for transdermally delivering a natriuretic peptide to a patient; comprising:
a microprojection member having a plurality of microprojections that are adapted to pierce the stratum corneum of the patient;
a solid state formulation disposed proximate said microprojection member; and
a hydrogel formulation, said hydrogel formulation adapted to communicate with said solid state formulation.

29. The delivery system of claim 28, wherein said solid state formulation is a solid film made by casting a liquid formulation comprising at least one natriuretic peptide, a polymeric material, a plasticizing agent, a surfactant and a volatile solvent.

30. The delivery system of claim 29, wherein said liquid formulation comprises 0.1-20 wt. % natriuretic peptide, 5-40 wt. % polymer, 5-40 wt. % plasticizer, 0-2 wt. % surfactant, and the balance comprising volatile solvent.

31. The delivery system of claim 29, wherein the concentration of said natriuretic peptide in said liquid formulation is in the range of approximately 0.1-2 wt. %.

32. The delivery system of claim 28, wherein the pH of said liquid formulation is below about pH 6.

33. A method of transdermally delivering a natriuretic peptide to a patient, comprising the steps of:
providing a microprojection member having a plurality of microprojections, said microprojection member having a coating disposed thereon, said coating including at least one natriuretic peptide;
applying said microprojection member to a skin site of said patient, whereby said plurality of microprojections pierce the stratum corneum and deliver said natriuretic peptide to said patient; and
removing said microprojection member from said skin site.

34. The method of claim 33, wherein said microprojection member remains applied to said skin site for a period of time in the range of 5 sec. to 24 hrs.

35. The method of claim 33, wherein said natriuretic peptide is selected from the group consisting of artrial natriuretic peptides (ANP), B-type natriuretic peptides (BNP), C-type natriuretic peptides and urodilatins, and analogs, active fragments, degradation products, salts and simple derivatives and combinations thereof.

36. The method of claim 33, wherein said natriuretic peptide comprises hBNP(1-32).

37. The method of claim 33, wherein said natriuretic peptide comprises in the range of approximately 1 μg-2000 μg of said biocompatible coating.

38. The method of claim 33, wherein said delivery of said natriuretic peptide exhibits improved pharmacokinetics compared to the pharmacokinetic characteristics of subcutaneous delivery.

39. A method for transdermally delivering a natriuretic peptide to a patient, comprising the steps of:
providing a microprojection assembly having a microprojection member and a gel pack, said microprojection member including a plurality of microprojections, said gel pack including a hydrogel formulation having at least one natriuretic peptide;
applying said microprojection member to a skin site of said patient, whereby a plurality of microslits are formed in the patient's stratum-corneum;
placing said gel pack on said microprojection member, whereby said hydrogel formulation is released from said gel pack and migrates into and through said microslits formed by said microprojections; and
removing said microprojection member from said skin site.

40. The method of claim 39, wherein said gel pack includes a release liner and said method includes the step of removing said release liner prior to placing said gel pack on said microprojection member.

41. The method of claim 39, wherein said microprojection member includes a biocompatible coating having at least one natriuretic peptide.

42. The method of claim 39, wherein said microprojection member remains applied to said skin site for a period of time in the range of 5 min. to 24 hrs.

43. The method of claim 39, wherein said natriuretic peptide is selected from the group consisting of artrial natriuretic peptides (ANP), B-type natriuretic peptides (BNP), C-type natriuretic peptides and urodilatins, and analogs, active fragments, degradation products, salts and simple derivatives and combinations thereof.

44. The method of claim 39, wherein said natriuretic peptide comprises hBNP(1-32).

45. The method of claim 39, wherein said natriuretic peptide comprises in the range of approximately 0.1-2 wt. % of said hydrogel formulation.

46. The method of claim 39, wherein said delivery of said natriuretic peptide exhibits improved pharmacokinetics compared to the pharmacokinetic characteristics of subcutaneous delivery.

47. A method for transdermally delivering a natriuretic peptide to a patient, comprising the steps of:
providing a microprojection assembly having a microprojection member and a gel pack, said microprojection member including a plurality microprojections, said microprojection member further including a biocompatible coating having at least one natriuretic peptide, said gel pack including a hydrogel formulation;
applying said microprojection member to a skin site of said patient, whereby a plurality of microslits are formed in the patient's stratum-corneum;
placing said gel pack on said microprojection member, whereby said hydrogel formulation is released from said gel pack and migrates into and through said microslits formed by said microprojections; and
removing said microprojection member from said skin site.

48. The method of claim 47, wherein said gel pack includes a release liner and said method includes the step of removing said release liner prior to placing said gel pack on said microprojection member.

49. The method of claim 47, wherein said microprojection member remains applied to said skin site for a period of time in the range of 5 min. to 24 hrs.

50. The method of claim 47, wherein said natriuretic peptide is selected from the group consisting of artrial natriuretic peptides (ANP), B-type natriuretic peptides (BNP), C-type natriuretic peptides and urodilatins, and analogs, active fragments, degradation products, salts and simple derivatives and combinations thereof.

51. The method of claim 47, wherein said natriuretic peptide comprises in the range of approximately 1 μg -2000 μg of said biocompatible coating.

52. The method of claim 47, wherein said natriuretic peptide comprises hBNP(1-32).

53. The method of claim 47, wherein said delivery of said natriuretic peptide exhibits improved pharmacokinetics compared to pharmacokinetics characteristic of subcutaneous delivery.

54. A method for transdermally delivering a natriuretic peptide to a patient, comprising the steps of:
providing a microprojection assembly having a microprojection member, a gel pack and a solid state formulation, said microprojection member including a plurality of microprojections, said gel pack including a hydrogel formulation, said solid state formulation being disposed proximate said microprojection member and including at least one natriuretic peptide;
applying said microprojection member to a skin site of said patient, whereby a plurality of microslits are formed in the patient's stratum-corneum;
placing said gel pack on said microprojection member, whereby said hydrogel formulation is released from said gel pack and migrates into and through said microslits formed by said microprojections; and
removing said microprojection member from said skin site.

55. The method of claim 54, wherein said gel pack includes a release liner and said method includes the step of removing said release liner prior to placing said gel pack on said microprojection member.

56. The method of claim 54, wherein said microprojection member remains applied to said skin site for a period of time in the range of 5 min. to 24 hrs.

57. The method of claim 54, wherein said natriuretic peptide is selected from the group consisting of artrial natriuretic peptides (ANP), B-type natriuretic peptides (BNP), C-type natriuretic peptides and urodilatins, and analogs, active fragments, degradation products, salts and simple derivatives and combinations thereof.

58. The method of claim 54, wherein said solid state formulation is formed from a liquid formulation having in the range of 0.1-2 wt. % of said natriuretic peptide.

59. The method of claim 54, wherein said natriuretic peptide comprises hBNP(1-32).

60. The method of claim 54, wherein said delivery of said natriuretic peptide exhibits improved pharmacokinetics compared to the pharmacokinetic characteristics of subcutaneous delivery.
